(19) United States
(12) Patent Application Publication
(10) Pub. No.: US 2012/0100526 A1
(43) Pub. Date: Apr. 26, 2012

(54) IDENTIFICATION AND DIFFERENTIATION OF NUCLEIC ACID SEQUENCE USING TEMPERATURE-DEPENDENT HYBRIDIZATION

(75) Inventors: John W. Czajka, West Grove, PA (US); Harry A. Glorikian, Arlington, MA (US)

(73) Assignee: SMITHS DETECTION INC., Edgewood, MD (US)

(21) Appl. No.: 13/139,004

(22) PCT Filed: Dec. 7, 2009
(86) PCT No.: PCT/US2009/066924

§ 371 Date: Aug. 2, 2011

Related U.S. Application Data
(60) Provisional application No. 61/193,609, filed on Dec. 10, 2008.

(51) Int. Cl. C12Q 1/68; C12Q 1/70; G01N 33/53

(52) U.S. Cl. 435/5; 436/501; 435/6.11

(57) ABSTRACT

Oligonucleotide probes are provided that are capable of hybridizing to different target nucleic acid sequences in a temperature-dependent manner allowing for detection of more than one target sequence by the same probe or by different probes having reporter labels with identical or similar detection characteristics. Also provided is a method for detecting target nucleic acid sequences using an oligonucleotide probe capable of hybridizing to the sequences in a temperature-dependent manner or using different probes having reporter labels with identical or similar detection characteristics.

### CROSS-REFERENCE TO RELATED PATENT APPLICATIONS

This application claims priority of U.S. Provisional Application No. 61/193,609, filed Dec. 10, 2008, which is hereby incorporated by reference.

### BACKGROUND

Nucleic acid amplification technologies exist that produce single stranded nucleic acid products. Detection of these amplification products typically requires a probe that will recognize a specific nucleic acid sequence with the amplified nucleic acid product (a “target sequence”). Such detection is often performed with an oligonucleotide probe that hybridizes to a specific target nucleic acid sequence under stringent conditions, allowing detection of the specific target sequence.

Stringent hybridization conditions, which are well-known in the art, are generally selected to be about 5° C. lower than the thermal melting point (Tₘ) for a specific sequence at a defined ionic strength and pH. The Tₘ is the temperature (under defined ionic strength and pH) at which 50% of the base pairs are dissociated. Although sequence dependent, stringent conditions typically will be those in which the salt concentration is at least about 1.5 to 6.0 mM at pH 7.0 to 8.3 and the temperature is between 40° C. and 72° C. When a sequence-specific probe is used, each target nucleic acid sequence generally requires a different probe with a different reporter label for each target sequence. The use of multiple probes having different target sequences and reporter labels can increase expense and result in complicated inter-probe interactions.

### SUMMARY

Accordingly, there remains a need in the art for a nucleic acid probe capable of identifying a plurality of amplification products in a temperature-dependent fashion.

To address these and other needs, oligonucleotide probes are provided that are capable of hybridizing to different nucleic acid target sequences in a temperature-dependent manner, allowing detection of more than one target nucleic acid sequence by the same probe.

One embodiment provides an oligonucleotide probe for detecting a target nucleic acid sequence, wherein the oligonucleotide probe hybridizes to a first nucleic acid sequence at a first temperature and a second nucleic acid sequence at a second temperature.

Another embodiment provides a method of detecting a two or more target nucleic acid sequences. The method comprises (a) contacting one or more target nucleic acid sequences with an oligonucleotide probe at a first temperature (b) altering the temperature to attain a second temperature, and (c) determining whether the probe hybridizes to a target nucleic acid sequence at the first temperature, the second temperature, or at both temperatures, wherein the first temperature can be correlated to a first target nucleic acid sequence and the second temperature can be correlated to the second nucleic acid sequence.

Another embodiment provides at least two oligonucleotide probes for detecting two target nucleic sequences, wherein each oligonucleotide has a reporter molecule having the same or similar detection characteristics, such as, for example, the same reporter molecule, but is designed to hybridize at different temperatures. One oligonucleotide probe can hybridize to a first target nucleic acid sequence at a first temperature and the second probe can hybridize to a second target nucleic acid sequence at a second temperature. Differentiation of the two hybridization events can be determined by an increase in reporter molecule signal at the temperature that associated the respective probe.

Another embodiment provides a method of detecting two or more target nucleic acid sequences with two or more oligonucleotide probes that are labeled with reporter molecules having similar or identical detection characteristics, such as, for example, the same reporter molecule, but are designed to hybridize to their respective targets at different temperatures. The method comprises (a) contacting one or more target nucleic acid sequences with a reporter-labeled oligonucleotide probe at a first temperature (b) altering the temperature to attain a second temperature, and (c) determining whether a second probe with the same reporter label hybridizes to its target nucleic acid sequence at the second temperature, wherein the signal at the first temperature can be correlated to a first target nucleic acid sequence and the signal at the second temperature can be correlated to the second nucleic acid sequence. In an embodiment, additional reporter-labeled probes can be used and each probe is capable of hybridizing to a target nucleic acid sequence at a defined temperature.

One embodiment provides a kit comprising an amplification reagent mixture containing at least one primer for amplifying one or more target nucleic acid sequences, and at least one oligonucleotide probe, wherein the oligonucleotide probe hybridizes to a first nucleic acid sequence at a first temperature and a second nucleic acid sequence at a second temperature A further embodiment provides a kit comprising an amplification reagent mixture containing at least one primer for amplifying one or more target nucleic acid sequences, and at least two oligonucleotide probes each having a reporter label having similar or identical detection characteristics, including, for example, the same reporter label, wherein a first oligonucleotide probe hybridizes to a first target nucleic acid sequence at a first temperature and a second probe to a second target nucleic acid sequence at a second temperature. The at least two oligonucleotide probes can be up to two, three, four, five, six, seven, eight, ten, or more than eleven, twelve, fifteen, or twenty probes.

In another embodiment, there is a probe, comprising an oligonucleotide probe for detection of a target nucleic acid sequence, wherein the oligonucleotide probe hybridizes to a first nucleic acid sequence at a first temperature and a to a second nucleic acid sequence at a second temperature.

In another embodiment, there is a method of detecting two or more target nucleic acid sequences, the method comprising:
a. contacting one or more target nucleic acid sequences with an oligonucleotide probe at a first temperature;
b. altering the temperature to attain a second temperature; and
c. determining whether the probe hybridizes to a target nucleic acid sequence at the first temperature, the second temperature, or at both temperatures.

In another embodiment, there is a method as described above and/or below, wherein the first temperature is correlated to a first target nucleic acid sequence and the second temperature is correlated to the second nucleic acid sequence.

In another embodiment, there is a method as described above and/or below, wherein the first temperature is correlateable to a first target nucleic acid sequence and the second temperature is correlateable to the second nucleic acid sequence.

In another embodiment, there is a kit comprising an amplification reagent mixture containing one or more pairs of primers for amplifying one or more target nucleic acid sequences, and at least one oligonucleotide probe, wherein the oligonucleotide probe hybridizes to a first nucleic acid sequence at a first temperature and to a second nucleic acid sequence at a second temperature.

In another embodiment, there is a composition comprising at least two oligonucleotide probes, wherein the oligonucleotide probes have a reporter label with identical and/or similar detection characteristics, wherein a first oligonucleotide probe hybridizes to a first target nucleic acid sequence at a first temperature, a second oligonucleotide probe hybridizes to a second target nucleic acid sequence at a second temperature, wherein the first target nucleic acid sequence is detected at a first temperature and the second nucleic acid sequence is detected at a second temperature, and wherein detection of the reporter label at the first temperature indicates presence of the first target nucleic acid sequence and detection of the reporter label at the second temperature indicates presence of the second target nucleic acid sequence.

In another embodiment, there is a method of detecting two or more target nucleic acid sequences with two or more oligonucleotide probes that are labeled with a reporter molecule having at least one of identical and similar detection characteristics comprising:

(i) contacting one or more target nucleic acid sequences with a reporter-labeled oligonucleotide probe at a first temperature;

(ii) altering the temperature to attain a second temperature;

(iii) determining whether a second probe with the reporter label hybridizes a target nucleic acid sequence at the second temperature.

In another embodiment as disclosed above and/or below, there is a method wherein the signal at the first temperature is correlateable to a first target nucleic acid sequence and the signal at the second temperature is correlateable to the second nucleic acid sequence.

In another embodiment as disclosed above and/or below, there is a method comprising correlating the signal at the first temperature to a first target nucleic acid sequence and correlating the signal at the second temperature to the second nucleic acid sequence.

In another embodiment, there is a kit comprising an amplification reagent mixture containing at least one primer for amplifying one or more target nucleic acid sequences, and at least two oligonucleotide probes having each having a reporter label having identical or similar detection characteristisc, wherein the first oligonucleotide probe hybridizes to a first nucleic acid sequence at a first temperature and the second probe hybridizes to a second nucleic acid sequence at a second temperature.

The above described and other features are exemplified by the following detailed description.

### DETAILED DESCRIPTION

It is useful to have an oligonucleotide probes that can hybridize to different amplification products in a temperature-dependent manner (“mismatch tolerant probes” or “temperature dependent probes”). Using mismatch-tolerant probes and measuring the hybridization of the probes at various temperatures, one determines the presence of a specific target sequence based on an a priori understanding of the target amplification sequences. Temperature-dependent probes can provide advantages for multiplex detection, identification, and discrimination of target nucleic acid sequences. Unless indicated otherwise, all technical and scientific terms are used in a manner that conforms to common technical usage. Generally, the nomenclature of this description and the described laboratory procedures, in cell culture, molecular genetics, and nucleic acid chemistry and hybridization, respectively, are well known and commonly employed in the art. Standard techniques are used for recombinant nucleic acid methods, oligonucleotide synthesis, microbial culture, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed, for example, in Sambrook et al., MOLECULAR CLONING A LABORATORY MANUAL, 2d ed. (Cold Spring Harbor Laboratory Press, 1989), and CURRENT PROTOCOLS IN MOLECULAR BIOLOGY, John Wiley & Sons, 1989). “Sequence identity” has an art-recognized meaning and can be calculated using published techniques. See COMPUTATIONAL MOLECULAR BIOLOGY, Lesk, ed. (Oxford University Press, 1988), BIOCOMPUTING: INFORMATICS AND GENOME PROJECTS, Smith, ed. (Academic Press, 1993), COMPUTER ANALYSIS OF SEQUENCE DATA, PART I, Griffin & Griffin, eds., (Humana Press, 1994), SEQUENCE ANALYSIS IN MOLECULAR BIOLOGY, Von Heinje ed., Academic Press (1987), SEQUENCE ANALYSIS PRIMER, Gribskov & Devereux, eds. (Macmillan Stockton Press, 1991), and Carillo & Lipton, SIAM J. Applied Math. 48: 1073 (1988). Methods commonly employed to determine identity or similarity between two sequences include but are not limited to those disclosed in GUIDE To HUGE COMPUTERS, Bishop, ed., (Academic Press, 1994) and Carillo & Lipton, supra. Methods to determine identity and similarity are codified in computer programs. Preferred computer program methods to determine identity and similarity between two sequences include but are not limited to the GCG program package (Devereux et al., Nucleic Acids Research 12: 387 (1984)), BLASTP, BLASTN, FASTA (Atschul et al., J. Mol. Biol. 215: 403 (1990)), and FASTDB (Brutlag et al., Comp. App. Biosci. 6: 237 (1990)).

One embodiment provides oligonucleotide probes for a nucleic acid amplification and detection assay capable of detecting and identifying target nucleic acids with variations in nucleotide sequence. These oligonucleotide probes can be mismatch tolerant probes and can hybridize to more than one target nucleic acid sequence in a temperature-dependent fashion. A second embodiment involve designing distinct probes that will hybridize to specific targets at different temperatures. This embodiment can be considered as multiplexing by hybridization temperature.

Oligonucleotide, as used herein, is generic and can be comprised of polydeoxyribonucleotides, polyribonucleotides, and any other type of polynucleotide which is an N-glycoside of a purine or pyrimidine base, or modified purine or pyrimidine base, including double- and single-stranded DNA, as well as double- and single-stranded RNA.

Oligonucleotide primers and probes are capable of hybridizing with a target nucleic acid sequence. “Hybridization” refers to the formation of a duplex structure by two single-stranded nucleic acids by complementary base pairing. Hybridization can occur between complementary nucleic acid strands or between nucleic acid strands that contain minor regions of mismatch. Conditions under which fully complementary nucleic acid strands will hybridize are referred to as “stringent hybridization conditions.” Two single-stranded nucleic acids that are complementary except for minor regions of mismatch are referred to as “substantially complementary”. Stable duplexes of substantially complementary sequences can be achieved under less stringent hybridization conditions. Those skilled in the art of nucleic acid technology can determine duplex stability empirically considering a number of variables including, for example, the length and composition of the oligonucleotides, ionic strength, and incidence and type of mismatched base pairs.

In one embodiment, a temperature-dependent probe hybridizes to two or more target nucleic acids with one target nucleic acid possessing at least 20%, at least 30%, at least 40%, at least 50%, at least 60%, at least 70%, at least 80, at least 90, at least 95, at least 96, at least 97, at least 98, or at least 99% sequence homology to one another.

Detection and Identification of amplified target nucleic acid sequences can be accomplished by contacting the amplified nucleic acid product with a mismatch-tolerant and/or temperature-dependent probes and detecting the formation of hybrid duplexes. Hybridization can occur at various temperatures. For example, hybridization can be performed at a temperature of from 10° C. to 90° C.

In one embodiment, one or more target nucleic acid sequences in a sample is amplified, hybridization is observed at a first temperature and then hybridization is observed at one or more temperatures that are different from the first temperature. Using a priori knowledge of the temperatures at which various target sequences would hybridize, one can determine which target nucleic acid sequences are present. In another embodiment the temperature is increased or decreased during the detection and identification step. Oligonucleotide probes can be used to detect target nucleic acids from any biological sample. Biological samples can include, for example, biological material of eukaryotic, prokaryotic, or viral origin.

Bacteria can be Gram negative, Gram positive bacteria, or Archaea. Gram negative bacteria include, for example and without limitation, Vibrio, Salmonella, Shigella, Pseudomonas, Escherichia, Klebsiella, Proteus, Enterobacter, Serratia, Moraxella, Legionella, Bordetella, Gardnerella, Haemophilus, Neisseria, Brucella, Yersinia, Pasteurella, Bacteroids, and Helicobacter. Gram positive bacteria include, for example, and without limitation, Bacillus, Clostridium, Arthrobacter, Micrococcus, Staphylococcus, Streptococcus, Listeria, Corynebacteria, Planococcus, Mycobacterium, Nocardia, Rhodococcus, and acid fast Bacilli such as Mycobacterium. Archaea bacteria include, for example, and without limitation Methanogens, halophiles, thermophiles, psychrophiles, crenarchaeota, euryarchaeota and, korarchaeota. In one embodiment, nanoemulsion compositions can be used to inactivate Bacillus, including, without limitation B. anthracis, B. cereus, B. circulans, B. subtilis, and B. megaterium. Nanoemulsion compositions can also be used to inactivate Clostridium, e.g., C. botulinum, C. perfringens, and C. tetani. Other bacteria that can be inactivated by a nanoemulsion include, but are not limited to, H. influenzae, N. gonorrhoeae, S. agalactiae, S. pneumonia, S. pyogenes and V. cholerae (classical and Eltor), and Yersinia, including, Y pestis, Y enterocolitica, and Y. pseudotuberculosis. In another embodiment, the bacteria is B. anthracis. In another embodiment, the bacteria is Mycobaterium tuberculosis.

Viruses that can be detected with a mismatch tolerant probe include, without limitation, any virus of the families Baculoviridae, Herpesviridae, Iridoviridae, Poxviridae, “African Swine Fever Viruses,” Adenoviridae, Caulimoviridae, Myoviridae, Phycodnaviridae, Tectiviridae, Papovaviridae, Circoviridae, Parvoviridae, Hepadnaviridae, Cystoviridae, Birnaviridae, Reoviridae, Coronaviridae, Flaviviridae, Togaviridae, “Arterivirus,” Astroviridae, Caliciviridae, Picornaviridae, Potyviridae, Retroviridae, Orthomyxoviridae, Filoviridae, Paramyxoviridae, Rhabdoviridae, Arenaviridae, and Bunyaviridae. In one embodiment, the virus is herpes, pox, papilloma, corona, influenza, hepatitis, sendai, sindbis and vaccinia viruses, west nile, hanta, and viruses which cause the common cold. In one embodiment, the probe detects coxsackievirus.

In one embodiment, the fungus is a yeast, such as, for example various species of Candida (e.g., Candida albicans) or filamentous yeast including but not limited to Aspergillus species or dermatophytes such as Trichophyton rubrum, Trichophyton mentagrophytes, Microsporum canis, Microsporum gypseum, and Epiderophyton floccosum, and types thereof, as well as others. Additional embodiments include eukaryotic organisms, which include, without limitation, protozoa, algae, plant, and animal nucleic acids.

Oligonucleotides can be of any suitable size, which depends on many factors, including the function or use of the oligonucleotide. Oligonucleotides can be prepared by any suitable method, including, for example, cloning, enzymatic restriction of larger nucleotides, and direct chemical synthesis by a method such as the phosphotriester method of Narang et al., Meth. Enzymol. 68:90-9 (1979), the phosphodiester method of Brown et al., Meth. Enzymol. 68:109-51 (1979), the diethylphosphoramidite method of Beaucage et al., Tetrahedron Lett. 22:1859-62 (1981), and the solid support method of U.S. Pat. No. 4,458,066. A review of synthesis methods is provided in Goodchild, Bioconjugate Chemistry 1:165-87(1990).

Mismatch-tolerant probes and/or temperature-dependent probes can be supplied in a kit. The kit can include some or all necessary reagents. The kit can include a single primer or one or more sets of primers and one or more oliglonucleotide probes.

The term “primer” refers to an oligonucleotide, whether natural or synthetic, capable of acting as an initiating point for DNA synthesis under conditions in which synthesis of a primer extension product complementary to a nucleic acid strand is induced. For example, such conditions include inclusion of four different nucleoside triphosphates and an agent for polymerization (i.e., DNA polymerase or reverse transcriptase) in an appropriate buffer and at a suitable temperature. A primer can be a single-stranded oligodeoxyribonucleotide. The length of a primer can vary and depends on the intended use of the primer. In one embodiment, a primer ranges from 6 to 40 nucleotides.

A primer need not reflect the exact sequence of the template, but should be sufficiently complementary to hybridize with a template. Primers can incorporate additional features which allow for the detection or immobilization of the primer, but do not alter the basic ability of the primer to act as a point of initiation of DNA synthesis.

The oligonucleotide primers and probes can be used in an amplification mixture. The terms “amplification reaction mixture” refers to a combination of suitable reagents for carrying out a nucleic amplification reaction. A reaction mixture typically consists of oligonucleotide primer(s), nucleotide triphosphates (dNTP), and a nucleic acid processing enzyme, such as, for example, a thermostable polymerase, a helicase, an RNA polymerase, or a ligase, in a suitable buffer. A suitable polymerase is described in, for example, U.S. Pat. No. 4,889,818. Any suitable helicase and RNA polymerase can be used and many are known in the prior art. Suitable helicase and RNA polymerase are described in, for example, European Patent No. 0629706 and U.S. Pat. No. 5,654,142.

Amplified nucleic acids can be detected using any suitable method. For example, in one embodiment, double-stranded DNA can be detected using non-sequence specific detection systems such as, YO-PRO®, YoYo® and SYBR® dyes, which exhibit more fluorescence when bound to double-stranded DNA, as compared to single-standed DNA.

In another embodiment, an oligonucleotide probe that is specific for a nucleic acid region, a “hybridization probe,” can be used. There are a number of different hybridization probes, however, the central feature of methods using hybridization probes is the identification of a nucleic acid present in a sample by detecting hybridization of an oligonucleotide probe to amplified target DNA or RNA.

Probes useful in nucleic acid hybridization techniques are capable of binding to a target nucleic acid of complementary sequence through one or more types of chemical bonds, usually through complementary base pairing via hydrogen bond formation. A probe may include natural bases (i.e., A, G, U, C or T) or modified bases (7-deazaguanosine, inosine, etc.). In addition, the nucleic acids can be joined by a linkage other than a phosphodiester bond, so long as it does not interfere with hybridization. Thus, probes can be peptide nucleic acids in which the constituent bases are joined by peptide bonds rather than phosphodiester linkages.

Oligonucleotide probes can be prepared by any means known in the art. The probes are preferably at least a 6,12, 14, 16, 18, 20, 22, 24, 30, or 40 nucleotides

An oligonucleotide probe optionally can be bound to a molecule which allows for the detection or immobilization of the probe, but does not alter the hybridization characteristics of the probe. One of skill in the art will recognize that, in general, the complement of an oligonucleotide probe is also suitable as a probe.

Probes complementary to defined target nucleic acid can be synthesized chemically, generated from longer nucleotides using restriction enzymes, or can be obtained using techniques such as polymerase chain reaction (PCR). The probes can be labeled, for example, with a radioactive, biotinylated, or fluorescent tag.

In one embodiment, sequence-specific probes can employ fluorescent resonance energy transfer (FRET) between two fluorescent dyes as the means of detection. Sequence-specific oligonucleotide probes are labeled with, for example, fluorescein, rhodamine and cyanine dyes using known labeling chemistry. An acceptor dye, such as DABCYL or methyl red, can be used as an acceptor (or quencher) dye.

In yet another embodiment, a molecular beacon, an oligonucleotide probe having a hairpin secondary structure and a donor-acceptor pair at the 5′ and 3′ end, can be generated with complementary sequences enabling the probe to bind to amplified nucleic acid regions. See, e.g. Tyagi and Kramer, Nat. Biotechnol. 14:303-8 (1996) and Tyagi et al., Nat. Biotechnol. 18:1191-6 (2000).

In an additional embodiment, an oligonucleotide probe as described in, for example, Lee et al., Anal. Chim. Acta 457:61-70 (2002), can be used to detect amplified nucleic acid regions.

One of skill in the art will recognize that the use of different detection assay labels or immobilization methods may require minor optimizations in conditions and/or probe sequences. The specific application will determine which probes are used.

An amplification reaction can further include an internal control for calibration and verification of the presence of target nucleic acid. Any suitable internal control can be used. Using an internal control, a duplexed amplification is run and the amplification products of both the target nucleic acid and the internal control nucleic acid are monitored.

An amplification reaction can also include primers and probes specific for other nucleic acids of interest. For example, an amplification reaction can be multiplexed to amplify and detect genes associated with other biological hazards.

Detection of amplification products can be conducted by any suitable method, such as end-point determination.

Oligonucleotides can be supplied as reagents contained in a system, such as a pre-packaged consumable. For example, a consumable can be in for form of a strip of material having a filter for capturing a biological particulate such as a nucleic acid. The strip can also contain other reagents for carrying out amplification and/or detection of a target nucleotide. The filter and the reagent can be disposed and extend longitudinally on the strip of material. In another embodiment, the oligonucleotides can be contained within a buffer container housing which is removably connected to a plunger housing. Using this device, a swab attached to an end of a plunger collects a sample of a specimen to be analyzed for biological warfare agents, such as ricin toxin. The swab and plunger are inserted into the plunger housing, a buffer container is positioned inside the buffer container housing and the buffer container housing and plunger housing are attached. A buffer passes through the swab and elutes off the sample and the sample mixes with a reagent. The prepared sample is transferred into a reaction tube by using a whipping action.

One or more mismatch-tolerant and temperature-dependent probes can be provided as part of a kit. A kit can include an amplification reagent mixture containing one or more pairs of primers for amplifying one or more target nucleic acid sequences. In some cases, oligonucleotide probes and/or primers can be supplied on an appropriate support membrane, such as a microwell plate, or lyophilized onto or within a consumable container, package or strip. Other optional components of a kit include, for example, an agent to catalyze the synthesis of primer extension products, substrate nucleoside triphosphates, appropriate buffers for amplification and hybridization reactions, and instructions for performing the detection method.

Mismatch-tolerant and/or temperature-dependent probes can be used to detect genetic material from microorganisms, including pathogenic microorganisms, plants, and animals. A pathogenic microorganism can be, without limitation, a bacteria, a virus, a fungus, a protozoan or a combination thereof.

In an exemplary embodiment, there is an oligonucleotide probe for detection of a target nucleic acid sequence, wherein the oligonucleotide probe hybridizes to a first nucleic acid sequence at a first temperature and a second nucleic acid sequence at a second temperature.

In another exemplary embodiment, there is a method of detecting a two or more target nucleic acid sequences, the method comprising:

a. contacting one or more target nucleic acid sequences with an oligonucleotide probe at a first temperature,

b. altering the temperature to attain a second temperature, and

c. determining whether the probe hybridizes to a target nucleic acid sequence at the first temperature, the second temperature, or at both temperatures,

wherein the first temperature can be correlated to a first target nucleic acid sequence and the second temperature can be correlated to the second nucleic acid sequence.

In another embodiment, there is a kit comprising an amplification reagent mixture containing one or more pairs of primers for amplifying one or more target nucleic acid sequences, and at least one oligonucleotide probe, wherein the oligonucleotide probe hybridizes to a first nucleic acid sequence at a first temperature and a second nucleic acid sequence at a second temperature.

In another embodiment, there is a composition comprising at least two oligonucleotide, probe wherein the oligonucleotide probes have a reporter label with identical or similar detection characteristics and wherein a first oligonucleotide probe hybridizes to a first target nucleic acid sequence at a first temperature, a second oligonucleotide probe hybridizes to a second target nucleic acid sequence at a second temperature, wherein the first target nucleic acid sequence is detected at a first temperature and the second nucleic acid sequence is detected at a second temperature, and wherein detection of the reporter label at the first temperature indicates presence of the first target nucleic acid sequence and detection of the reporter label at the second temperature indicates presence of the second target nucleic acid sequence.

In another embodiment, there is a method of detecting two or more target nucleic acid sequences with two or more oligonucleotide probes that are labeled with a reporter molecule having identical or similar detection characteristics comprising:

(i) contacting one or more target nucleic acid sequences with a reporter-labeled oligonucleotide probe at a first temperature

(ii) altering the temperature to attain a second temperature, and

(iii) determining whether a second probe with the reporter label hybridizes a target nucleic acid sequence at the second temperature, wherein the signal at the first temperature can be correlated to a first target nucleic acid sequence and the signal at the second temperature can be correlated to the second nucleic acid sequence.

In another embodiment, there is a kit comprising an amplification reagent mixture containing at least one primer for amplifying one or more target nucleic acid sequences, and at least two oligonucleotide probes having each having a reporter label having identical or similar detection characteristics, wherein the first oligonucleotide probe hybridizes to a first nucleic acid sequence at a first temperature and the second probe to a second nucleic acid sequence at a second temperature.

Given the disclosure herein, one versed in the art would appreciate that there are other embodiments and modifications within the scope and spirit of the present invention. Accordingly, all modifications attainable by one versed in the art from the present disclosure within the scope and spirit of the present invention are to be included as further embodiments of the present invention.

1. (canceled)

2. A method of detecting two or more target nucleic acid sequences, the method comprising:
a. contacting one or more target nucleic acid sequences with an oligonucleotide probe at a first temperature;
b. altering the temperature to attain a second temperature; and
c. determining whether the probe hybridizes to a target nucleic acid sequence at the first temperature, the second temperature, or at both temperatures.

3. The method of claim 2, wherein the first temperature is correlated to a first target nucleic acid sequence and the second temperature is correlated to the second nucleic acid sequence.

4. The method of claim 2, wherein the first temperature is correlateable to a first target nucleic acid sequence and the second temperature is correlateable to the second nucleic acid sequence.

5.-6. (canceled)

7. A method of detecting two or more target nucleic acid sequences with two or more oligonucleotide probes that are labeled with a reporter molecule having at least one of identical or similar detection characteristics comprising:
contacting one or more target nucleic acid sequences with a oligonucleotide probe, that has a reporter-label, at a first temperature;
(ii) altering the first temperature to attain a second temperature;
(iii) determining whether a second probe with the same type of reporter₌label hybridizes a target nucleic acid sequence at the second temperature.

8. The method of claim 7, wherein the signal at the first temperature is correlateable to a first target nucleic acid sequence and the signal at the second temperature is correlateable to the second nucleic acid sequence.

9. The method of claim 7, further comprising correlating the signal at the first temperature to a first target nucleic acid sequence and correlating the signal at the second temperature to the second nucleic acid sequence.

10. (canceled)

11. The method of claim 2, wherein the first and second temperatures are between 10 and 90 degrees Celsius.

12. The method of claim 2, wherein the oligonucleotide probe comprises a molecular beacon.

13. The method of claim 9, wherein the first and second nucleic acid sequences posses at least 95% homology to each other.

14. The method of claim 7, wherein the first and second temperatures are between 10 and 90 degrees Celsius.

15. The method of claim 7, wherein the first reporter-labeled oligonucleotide probe comprises a molecular beacon.

16. A method of detecting target nucleic acid comprising:
a) combining, at a first temperature, in a mixture suspected of containing first and second target nucleic acid sequences:
i) a first oligonucleotide probe including a first label that provides a first detectable signal, that can be correlated to the first target nucleic acid sequence's presence, when hybridized to the first target nucleic acid sequence, and
ii) a second oligonucleotide probe including a second label that provides a second detectable signal, that can be correlated to the second target nucleic acid sequence's presence, when hybridized to the second target nucleic acid sequence;
b) altering the mixture's temperature to a second temperature;
c) determining, at the second temperature, whether the second probe hybridizes to the second target nucleic acid sequence.

17. The method of claim 16, wherein the first and second labels have similar or identical detection characteristics.

18. The method of claim 16, wherein the first and second labels comprise a same type label.

19. The method of claim 16, further comprising determining whether the first probe hybridizes to the first target nucleic acid sequence at the first temperature.

20. The method of claim 16, wherein the first and second target nucleic acid sequences posses at least 95% homology to each other.

21. The method of claim 16, wherein the first and second target nucleic acid sequences posses at least 98% homology to each other.

22. The method of claim 16, wherein the first oligonucleotide probe comprises a molecular beacon.

23. The method of claim 16, wherein the second oligonucleotide probe comprises a molecular beacon.
